(19) United States
(12) Patent Application Publication
(10) Pub. No.: US 2022/0409871 A1
(43) Pub. Date: Dec. 29, 2022

(54) SYSTEM AND MINIATURE DEVICES FOR DELIVERING A THERAPEUTIC COMPONENT TO A TREATMENT SITE IN A PATIENT

(71) Applicant: BIONAUT LABS LTD, Herzliya (IL)

(72) Inventors: Michael SHPIGELMACHER, Los Angeles, CA (US); Alex KISELYOV, San Diego, CA (US); Florent CROS, Los Angeles, CA (US); Darrell HARRINGTON, Canoga Park, CA (US); Suehyun CHO, Los Angeles, CA (US); John CAPUTO, Los Angeles, CA (US)

(73) Assignee: BIONAUT LABS LTD, Herzliya (IL)

(21) Appl. No.: 17/772,999

(22) PCT Filed: Nov. 4, 2020
(86) PCT No.: PCT/US20/58964

§ 371 Date: Apr. 28, 2022

Related U.S. Application Data
(60) Provisional application No. 62/930,863, filed on Nov. 5, 2019.

(51) Int. Cl. A61M 31/00; A61K 45/06

(52) U.S. Cl. CPC A61M 31/002; A61K 45/06; A61M 2205/3515

(57) ABSTRACT

A miniature device is provided for use in a system configured to deliver a therapeutic component to a treatment site in a patient. The miniature device comprises at least one steering portion comprising a magnetic material, and at least one carrier portion affixed to the steering portion and comprising the therapeutic component. The carrier portion is configured to at least partially dissipate under one or more predetermined conditions at the treatment site, thereby releasing the therapeutic component. Further provided is a system comprising one or more such miniature devices and a magnetic inducing apparatus configured to be operated to generate a varying magnetic field, thereby remotely controlling motion of the miniature device.

### FIELD OF THE INVENTION

The presently disclosed subject matter relates to systems and miniature device configured to navigate within a patient to deliver a payload to a predetermined location therewithin, and in particular to such systems which use magnetic fields to direct operation of miniature devices within a patient.

### BACKGROUND

Remote control of medical devices moving inside the human body can be useful for a variety of purposes, including delivery of therapeutic payloads, diagnostics or surgical procedures. Such devices may include microscale or nanoscale robots, medical tools, “smart pills,” etc. Such devices may be able to move in the body either through self-propulsion or an external propulsion mechanism. Accurate location and tracking of such devices may be necessary to ensure their proper functioning at the right anatomical location, and more specifically accurate delivery of the therapeutic payloads and/or diagnostics substances.

### SUMMARY

According to an aspect of the presently disclosed subject matter, there is provided a miniature device for use in a system configured to deliver a therapeutic component to a treatment site in a patient, the miniature device comprising:
at least one steering portion comprising a magnetic material; and
at least one carrier portion affixed to the steering portion and comprising the therapeutic component, the carrier portion being configured to at least partially dissipate under one or more predetermined conditions at the treatment site, thereby releasing the therapeutic component.

The carrier portion may further comprise a binder material mixed with the therapeutic component and being configured to undergo the dissipation.

The binder material may comprise a biodegradable and/or a bioerodible polymer.

The binder material may comprise one or more selected from the group including polylactic acid, agar, poly(lactic-co-glycolic acid), chitosan, hyaluronic acid, a hyaluronic acid salt, gelatin, glucose, and carboxymethyl cellulose.

The miniature device may further comprise an auxiliary carrier portion configured to at least partially dissipate under one or more predetermined conditions at the treatment site.

The auxiliary carrier portion may completely surround the carrier portion.

The auxiliary carrier portion may comprise a therapeutic component which differs from that of the carrier portion.

The auxiliary carrier portion may comprise the same therapeutic component as does the carrier portion at a different concentration.

The auxiliary carrier portion may be free of a therapeutic component.

The carrier portion may be formed with one or more channels open at an outer surface thereof and extending therewithin.

The carrier portion may be formed with one or more chambers therewith. At least one of the chambers may be evacuated. At least one of the chambers may comprise therewithin one or more gases selected from the group including air, hydrogen, oxygen, nitrogen, and carbon dioxide.

The carrier portion may be affixed to the steering portion by an adhesive material.

The adhesive material may be configured to be disrupted under a predetermined condition, thereby separating the carrier portion from the steering portion. The predetermined condition under which the adhesive material is configured to be disrupted may be one or more selected from the group including melting, dissolving in a solvent, chemically induced matrix rupture, exposure to radio and/or ultrasound waves, exposure to near infrared frequency.

The adhesive material may be insulated from the environment by a bioerodible material configured to delay the disruption of the adhesive material.

The carrier portion may surround the steering portion.

The steering portion may comprise a non-magnetic shell at least partially surrounding the magnetic material, the carrier portion being at least partially affixed thereto.

The steering portion may comprise two magnets constituting the magnetic material and being spaced along a longitudinal axis of the miniature device, the steering portion further comprising a non-magnetic bridging member spanning therebetween.

The carrier portion may be disposed surrounding the bridging member.

The vectors of the magnetic moments of the magnets may be parallel, antiparallel, or perpendicular to each other.

The magnets may be oriented such that the vectors of their magnetic moments are perpendicular or parallel to the longitudinal axis of the miniature device.

The miniature device may be shaped substantially as a prolate spheroid.

The miniature device may be formed with an indentation at a rear end thereof, the indentation being configured to accommodate a front end of another similarly formed miniature device.

The steering portion may comprise a tube made of an elastomeric materiel and being formed with one or more through-going apertures, the carrier portion being disposed within the tube and having a larger diameter than the tube.

The steering portion may further comprise a magnet closing each end of the tube.

The tube may be magnetic.

The carrier portion may comprise a liquid and a rigid casing therearound, the rigid casing being configured to undergo the dissipation.

The steering portion may be disposed within the liquid.

The carrier portion may comprise one or more materials configured to effervesce during the dissipation.

According to an aspect of the presently disposed subject matter, there is provided a system configured to deliver a therapeutic component to a treatment site in a patient, the system comprising at least one miniature device as described above with respect to the previous aspect, the system further comprising a magnetic inducing apparatus configured to be operated to generate a varying magnetic field, thereby remotely controlling motion of the miniature device.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 schematically illustrates a system for delivering a therapeutic component to a treatment site in a patient's body;

FIG. 2 illustrates a miniature device of the system illustrated in FIG. 1;

FIGS. 3 through 6 are schematic cross-sectional views of different examples of a miniature device of the system illustrated in FIG. 1;

FIGS. 7A and 7B, illustrate, respectively, an example of the miniature device of the system illustrated in FIG. 1 before and after disruption of an adhesive material thereof;

FIG. 8 is a schematic cross-sectional view of an example of a miniature device of the system illustrated in FIG. 1, comprising cavities in a carrier portion thereof;

FIG. 9 is a schematic cross-sectional view of an example of a miniature device of the system illustrated in FIG. 1, comprising an auxiliary carrier portion;

FIGS. 10 and 11 are schematic cross-sectional views of different examples of a miniature device of the system illustrated in FIG. 1

FIGS. 12A through 12D schematically illustrate separation of steering and carrier portions of a miniature device of the system illustrated in FIG. 1 according to some examples of the presently disclosed subject matter;

FIGS. 13A is a perspective view of a miniature device of the system illustrated in FIG. 1 according to some examples of the presently disclosed subject matter;

FIG. 13B is a cross-sectional view taken along line III-III in FIG. 13A;

FIG. 14 is a schematic cross-sectional view of two miniature devices of the system illustrated in FIG. 1 according to some examples of the presently disclosed subject matter, arranged in a procession;

FIG. 15A is a perspective view of another example of a miniature device of the system illustrated in FIG. 1;

FIG. 15B is a cross-sectional view taken along line V-V in FIG. 15A;

FIG. 15C is a perspective view of a steering portion of the miniature device illustrated in FIG. 15A;

FIG. 16 is a cross-sectional view of a modification of the miniature device illustrated in FIG. 15A;

FIGS. 17A and 17C are side views of examples of steering portions of miniature devices of the system illustrated in FIG. 1;

FIGS. 17B and 17D are front views of the steering portions illustrated in, respectively, FIGS. 17A and 17C;

FIG. 18A is a perspective view of another example of a miniature device of the system illustrated in FIG. 1, in a constricted state thereof;

FIG. 18B is a cross-sectional view of the miniature device illustrated in FIG. 18A, in a bulging state thereof;

FIG. 18C is a perspective view of a modification of the miniature device illustrated in FIG. 18A, in a constricted state thereof;

FIG. 19 is a cross-sectional view of another modification of the miniature device illustrated in FIG. 18A, in a bulging state thereof; and

FIG. 20 is a perspective view of another example of a miniature device of the system illustrated in FIG. 1.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

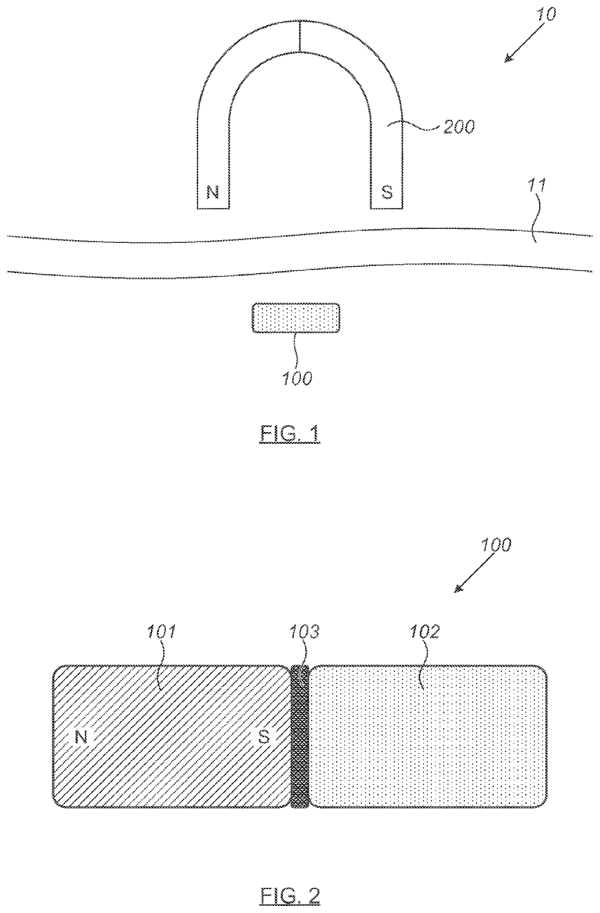

As illustrated in FIG. 1, there is provided a system 10 configured to facilitate delivery of one or more chemical compounds of medicinal, diagnostic, evaluative, and/or therapeutic relevance, one or more small molecules, biologics, cells, one or more radioisotopes, one or more vaccines, etc. (hereinafter “therapeutic component”), for example via body fluids, an anatomic lumen, and/or soft tissue, to a predetermined location within a patient's body (hereinafter “treatment site”). According to some examples, the therapeutic component may comprise one or more mechanical devices.

The system 10 comprises a miniature device 100 and a magnetic inducing apparatus, schematically indicated at 200, configured to control the miniature device. The miniature device 100 is configured to carry the therapeutic component. The magnetic inducing apparatus 200 is configured to be operated to generate a varying magnetic field and thereby remotely, i.e., from a location exterior to a patient's body 11, control the motion of the miniature device 100 within the body.

According to some embodiments, characteristics of the magnetic field, for example including, but not limited to, distance, directionality, intensity, gradient, time dependence/independence, etc., may be controlled by a user in order to remotely control the motion of the device 100.

According to some embodiments, for example as illustrated in FIG. 2, the miniature device 100 comprises a magnetic steering portion 101 composed partially or entirely of a magnetic material, and a carrier portion 102 affixed thereto.

The steering portion 101 is configured to interact with the magnetic field generated by the magnetic inducing apparatus 200, thereby facilitating control of the miniature device thereby.

The carrier portion 102 comprises, partially or in totality, one or more therapeutic components. It may further comprise a binder material carrying the therapeutic component, e.g., mixed therewith.

According to some embodiments the carrier portion 102, for example the binder material thereof, is configured to dissipate, thereby releasing the therapeutic component therefrom. The dissipation may be effected by any suitable means, including, but not limited to, dissolving, being broken apart, disintegrating, etc. The dissipation may occur either automatically upon contact with a liquid, such as bodily fluid occurring at the treatment site, for example at pace suitably slow to allow the miniature device 100 to be brought to the treatment site, or upon a directed external action. The dissipation may be induced by any suitable means, for example by exposure to electromagnetic radiation within a specific range, for example radio waves, near infrared, etc., acoustical waves such as an ultrasound signal, chemically induced matrix rupture, or dissolving in a solvent such as water or a bodily fluid such as blood, plasma, lymph, bile, or cerebrospinal fluid.

According to some embodiments, the binder material of the carrier portion 102 is configured to dissolve in bodily fluid over time, following a predictable pace. According to some embodiments, the binder material comprises a biodegradable and/or bioerodible polymer, including, but not limited to, polylactic acid, agar, poly(lactic-co-glycolic acid), chitosan, hyaluronic acid and its salts, gelatin with/without additives, glucose, and/or carboxymethyl cellulose, and any combinations thereof. According to some embodiments, the bioerodible polymer undergoes a predictable decomposition by hydrolysis in the biological compartment of interest. In some embodiments, this decomposition occurs in seconds, minutes, hours, days, or months, depending on the nature of the polymer, and/or internal/external conditions.

The carrier portion 102 may be connected to the steering portion 101 in any suitable manner. According to some examples, the steering portion 101 may comprise a permanent magnet and/or an electromagnet (e.g., comprising a power source and/or being configured to be powered externally, for example using wireless power transfer such as inductive charging) and be attached to the carrier portion 102 using an adhesive material 103 as illustrated in FIG. 2, and or being at least partially enclosed therewithin, for example as described below. According to some examples (not illustrated), the steering portion 101 comprises a plurality of magnetic particles, such as nanoparticles and/or microparticles, dispersed in a polymer matrix. According to some embodiments, the polymer matric comprises an elastomer.

It will be appreciated that while FIG. 2, as well as some of the subsequent figures, indicates magnetic polarity of the steering portion 101, this is done for the sake of illustration only, e.g., to more clearly indicate the magnetic properties thereof, this is not to be construed as limiting. In practice, the steering portion 101 may have a magnetic polarity which differs from that indicated, and/or may have no polarity at all, for example comprising a ferromagnetic material which is not magnetized prior to exposure to the magnetic field produced by the magnetic inducing apparatus 200.

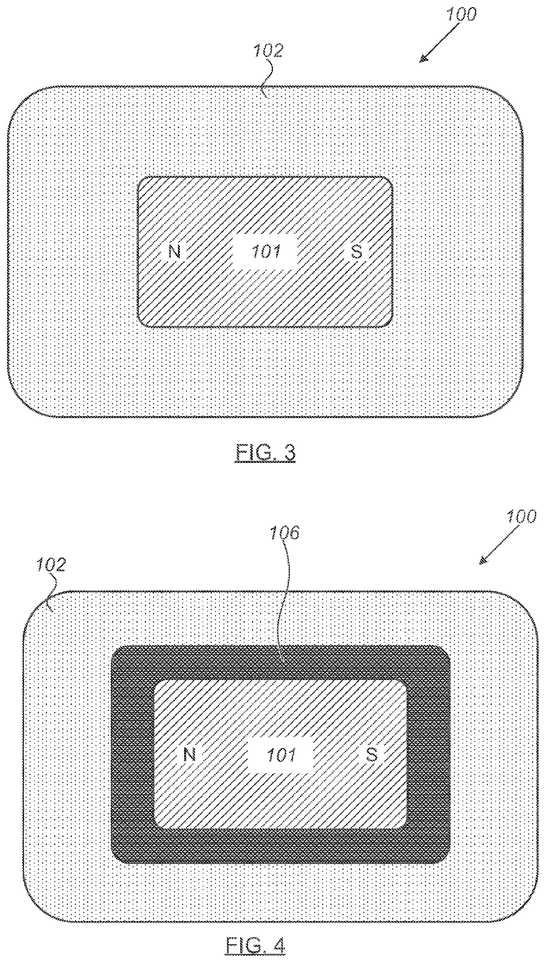

According to some embodiments, and as demonstrated in FIG. 3, the carrier portion 102 at least partially surrounds the steering portion 101.

According to some embodiments, and as illustrated in FIGS. 4 through 6, the steering portion 101 comprises a protective shell 106, e.g., being made of a non-magnetic material, at least partially surrounding the magnetic portion thereof. The carrier portion 102 may be attached, partially or entirely, to the protective shell 106. According to some embodiments, the shell 106 is made of Teflon. It will be appreciated that herein the specification and appended claims, the term “shell” is to be construed in its broadest sense, including, but not limited to shells, coatings, etc.

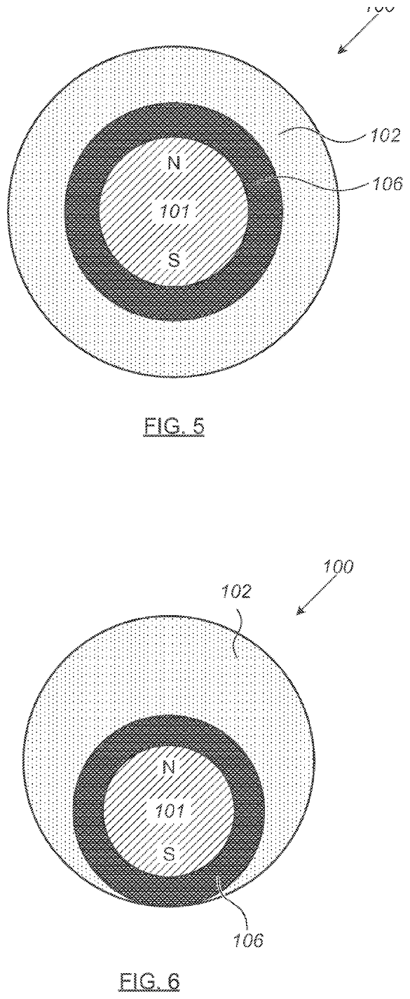

The steering portion 101 may be of any suitable shape, for example being spherical or cylindrical, as illustrated, inter alia, in FIGS. 5 and 6.

As illustrated in FIG. 6, the miniature device 100, for example including the shell 106, may be disposed eccentrically with the carrier portion 102, i.e., the steering portion and its shell are mounted substantially closer to the outside surface of the carrier portion 102 than to its center, thereby giving rise to an asymmetry in the miniature device's 100 design. According to some examples, a miniature device 100 having such an asymmetry may be induced to undergo undulating, wobbling, wiggling, etc., by suitably varying the magnetic field produced by the magnetic inducing apparatus 200.

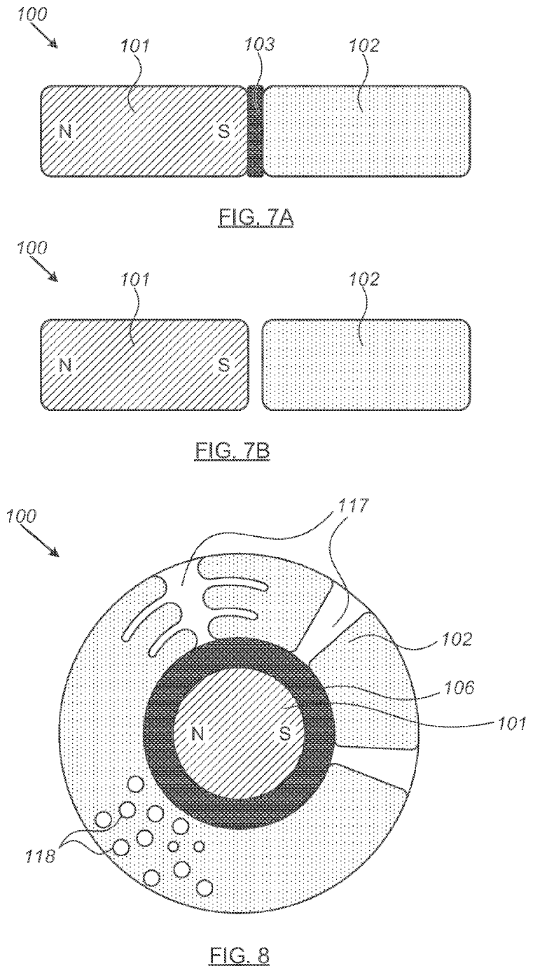

According to some embodiments, for example as illustrated in FIGS. 7A and 7B, the adhesive material 103 are configured to be disrupted, such that the carrier portion 102 is separated from the steering portion 101, for example using one or more of a variety of disrupting means, including, but not limited to, melting, dissolving in a solvent, chemically induced matrix rupture, exposure to radio and/or ultrasound waves, exposure to near infrared frequency, etc. Examples of solvents may include, but are not limited to, water, body fluids such as blood, plasma, lymph, bile, or cerebrospinal fluids.

As illustrated in FIG. 8, according to some embodiments the carrier portion 102 is formed with cavities, such as channels 117 open at an outer surface thereof and/or chambers 118, the cavities being configured to ease the ingress of the solvent and to hasten the dissipation thereof. According to some examples, some or all of the chambers 118 are evacuated. According to other examples, some or all of the chambers 118 are filled with a gas, which may include, but is not limited to, air, hydrogen, oxygen, nitrogen, carbon dioxide, and/or any combination thereof. According to other examples, some or all of the chambers 118 comprise a material therewithin, such as a compound or polymer, which differs from that of the carrier portion 102. The gas and/or material within the chambers 118 may be selected based on the nature of its reaction with the surrounding environment (e.g., a bodily fluid), which may differ from that of the binder material of the carrier portion 102. According to some embodiments, the gas and/or material within the chambers 118 may be configured to hasten the dissipation of the carrier portion 102, for example in a desirable, predictable, and/or controllable manner.

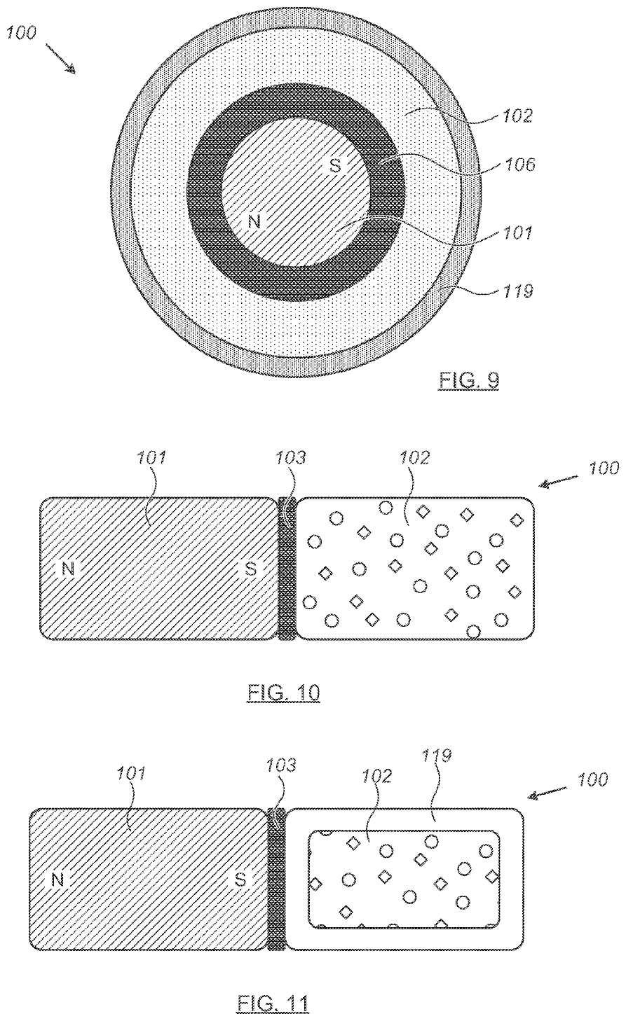

As illustrated in FIG. 9, the miniature device 100 may comprise an auxiliary carrier portion 119, for example surrounding the carrier portion 102. The auxiliary carrier portion 119 may be provided according to any one or more of the examples described above, mutatis mutandis. The auxiliary carrier portion 119 may comprise a binder material mixed with the same therapeutic component as is in the carrier portion 102 at a higher or lower concentration, it may comprise no therapeutic component (still referred to herein as an “auxiliary carrier portion” for the sake of simplicity), or it may comprise a different therapeutic component. Moreover, the binder material of the auxiliary carrier portion 119 may be different than that of the carrier portion 102. According to some embodiments the miniature device 100 is configured to be introduced in a living organism and driven using the magnetic inducing apparatus 200 to the treatment site and exposed to the internal or external release stimuli for the duration of time for the layer 119 to dissolve. It will be appreciated that more than one auxiliary carrier portions 119 may be provided, mutatis mutandis.

According to some embodiments, for example as illustrated in FIG. 10, the carrier portion 102 comprises a monomolecular, binary, or more complex chemical mixture designed to initiate a chemical reaction as exemplified by evolution of gas or heat, breakage of a chemical bond upon exposure to internal or external stimuli. In a representative but non-limiting example, a dry powder of citric acid and sodium bicarbonate (e.g., having a 1:1 mole ratio) that react in presence of an aqueous solution and result in effervescence to produce CO₂ that acts as a matrix disruptor.

According to some embodiments, for example as illustrated in FIG. 11, the carrier portion 102 comprises dry powder of citric acid and sodium bicarbonate (e.g., having a 1:1 mole ratio) that is designed to react in the presence of an aqueous solution and result in effervescence. The carrier portion 102 is surrounded by an auxiliary carrier portion 119, comprising a bioerodible binder material. The auxiliary carrier portion 119 temporarily insulates the carrier portion 102 from the aqueous solution. According to some embodiments, the chemical make-up and thickness of the auxiliary carrier portion 119 are selected so as to partially or totally dissolve in a predictable average time.

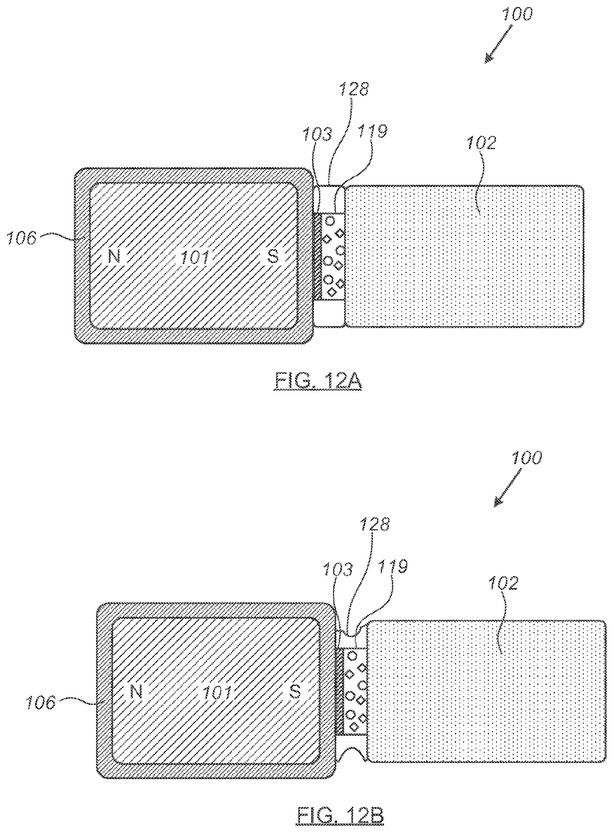

According to some embodiments, for example as illustrated in FIGS. 12A through 12D, the steering portion 101 is composed, partially or entirely, of a magnetic material and is optionally surrounded by a protective shell 106. According to some embodiments, the carrier portion 102 is composed, partially or in totality, of a chemical compound constituting the therapeutic component, and is affixed to the auxiliary carrier portion 119, which comprises one or more substances, e.g., citric acid and sodium bicarbonate, which react in the presence of an aqueous solution resulting in effervescence. The adhesive 103 is disposed so as to affix the auxiliary carrier portion 119 to the shell 106. A protective element 128 is provided, configured to temporarily insulate the auxiliary carrier portion 119 from the environment. According to some embodiments, the protective element 128 is bioerodible and begins to erode or dissolve when brought in contact with bodily fluids, as illustrated in FIG. 12B. Once some or all of element 128 erodes, the auxiliary carrier portion 119 is exposed to the bodily fluids and reacts, e.g., in an effervescent fashion, thereby forcing the steering portion 101 and the carrier portion 102 apart. As a result, as illustrated in FIG. 12D, the carrier portion 102 detaches from the steering portion 101, which is free to be directed away from the site of interest, leaving the carrier portion 102 in place. For example, it may be retrieved under direction of the magnetic inducing apparatus 200, using a surgical procedure, or excreted using physiologically relevant biofluid flow, e.g., bile, urine, etc. The retrieved steering portion 101 may be subjected to a suitable sterilization protocol and be reused.

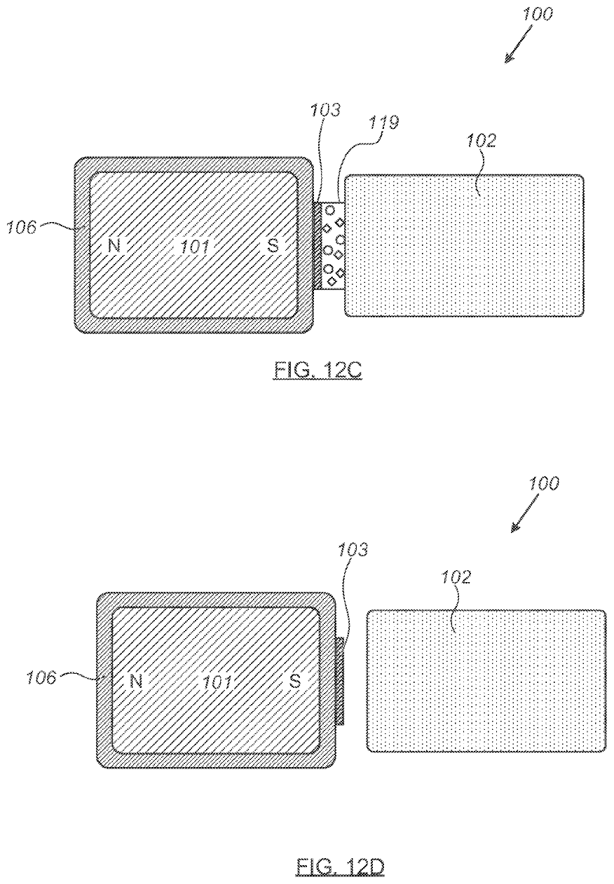

According to some embodiments, for example as illustrated in FIGS. 13A and 13B, the steering portion 101 comprises two magnets 140 partially or completely surrounded by a shell 106 having, with one or more cutouts 120 therewithin, giving rise to a bridging member 141 spanning therebetween. Each of the cutouts 120 is filled with a carrier portion 102, for example giving rise to a generally spheroidal shape, e.g., shaped generally as a prolate spheroid, of the miniature device 100. The generally spheroidal shape of the miniature device 100 may facilitate reliable passage thereof through the body, e.g., through central nervous system compartments such as portions of the brain and the spine.

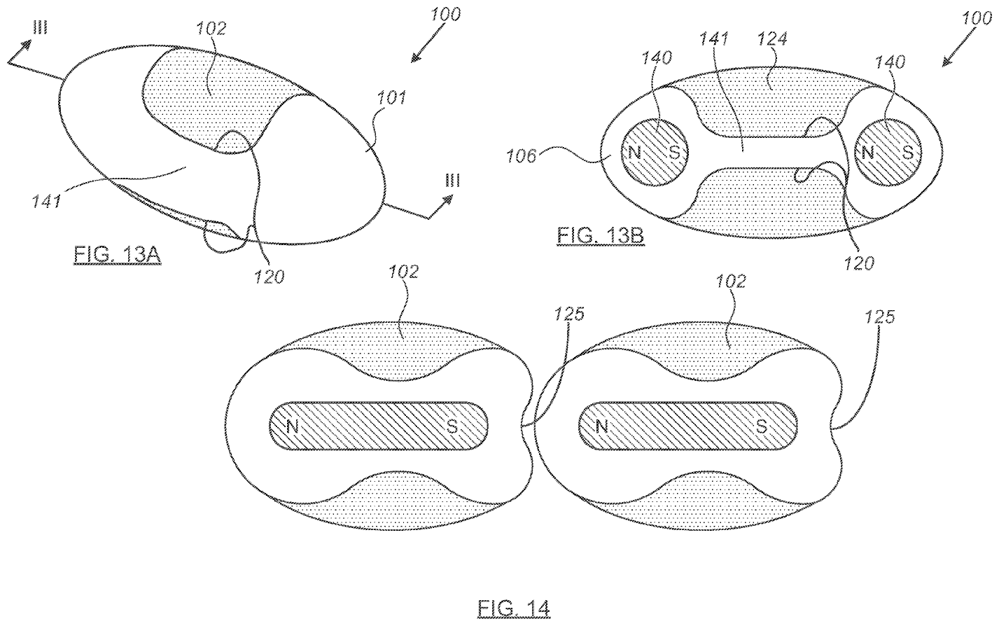

According to some embodiments, for example as illustrated in FIG. 14, a plurality of miniature devices 100 (two shown) are provided, each formed so as to cooperate with other of the miniature devices to facilitate aligning themselves into a suitable arrangement when brought into proximity with one another, for example a linear procession. For example, each of the miniature devices 100 may be formed generally as an ellipsoid, for example as described above with reference to and as illustrated in FIGS. 13A and 13B, and being formed with an indentation 125 at a rear end thereof. When two of the miniature devices 100 are brought into proximity with one another, the rounded front end of one of them is magnetically attracted to the rear end of another, and is accommodated within the indentation 125. It will be appreciated that while two miniature devices 100 are so illustrated in FIG. 14, any suitable number of miniature devices may be so arranged to produce a procession of any suitable length. It will be appreciated that while the miniature devices illustrated in FIG. 14 each comprise a single magnet, some or all may each be provided with two magnets, for example as described above with reference to and as illustrated in FIGS. 13A and 13B, mutatis mutandis.

A plurality of miniature devices 100 as described above with reference to and as illustrated in FIG. 14 may be used to control delivery of a one or more therapeutic compounds to a treatment site. According to some examples, the rate at which the therapeutic compound is delivered to a treatment site is controlled by the speed at which the procession of such miniature devices 100 moves theretoward. According to some examples, such a procession may allow a user to vary the dosage, rate of delivery, type of therapeutic compound being delivered during a procedure, e.g., adding one or more miniature devices 100 to the procession whose therapeutic compound has antifibrinolytic properties. Similarly, the amount of therapeutic compound delivered to a treatment site may be thus more evenly spread out over a predetermined span of time. According to some examples, different therapeutic compounds may be delivered to a treatment site in a predetermined sequence, each of which is carried by one or more different miniature devices in the procession.

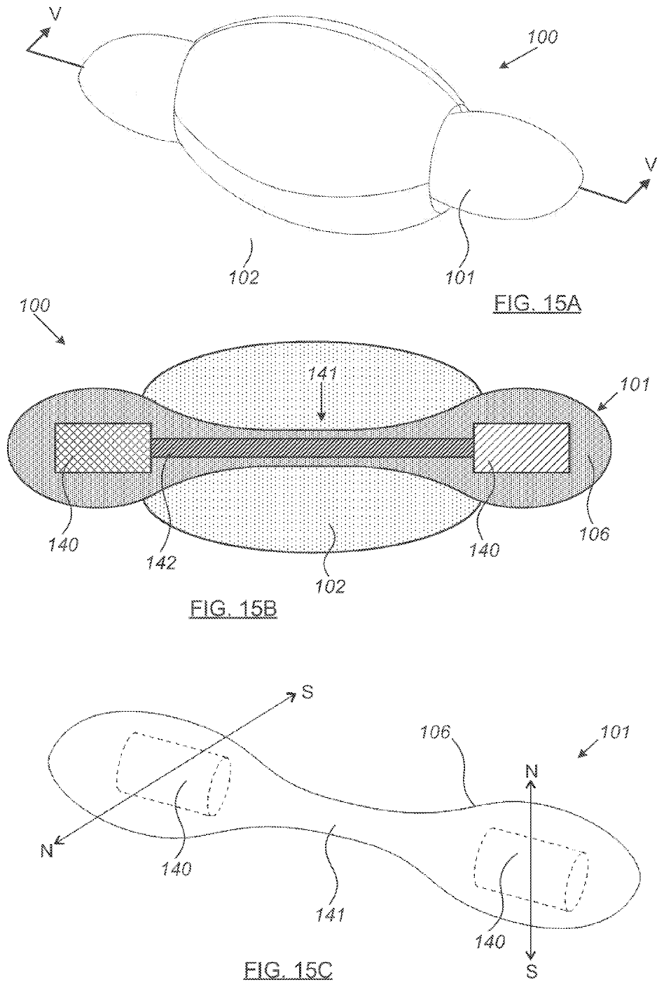

According to some embodiments, for example as illustrated in FIGS. 15A and 15B, the steering portion 101 may comprise two magnets 140, each for example being a permanent magnet and/or an electromagnet as described above, disposed at opposite ends of the miniature device 100, and connected by a non-magnetic bridging member, generally indicated at 141, extending along a longitudinal axis of the miniature device. The bridging member 141 has a radius which is smaller than that of the front and rear ends of the steering portion. According to some embodiments, the magnets 140 are disposed within a non-magnetic shell 106, which comprises at least a portion of the bridging member 141. The shell 106 may be rigid or flexible, for example being made of an elastomer. According to some embodiments, a linking element 142, such as a flexible truss, is provided spanning between the magnets 140 and constituting at least a portion of the bridging member 141. The carrier portion 102 is formed with a through-going aperture 144, accommodating the bridging member 141 therethrough. The radius of the through-going aperture 144 is smaller than that of the front and rear ends of the steering portion 101. This arrangement ensures that the carrier portion 102 is maintained on the steering portion 101 until it dissipates, for example as described above.

According to some embodiments, each of the magnets 140 is oriented such that the vector of its magnetic moment (i.e., the orientation of its north and south poles) is perpendicular to the longitudinal axis of the miniature device 100. According to some embodiments, for example as illustrated in FIG. 15C (in which the magnets 140 within the shell 106 are shown in broken lines) the vectors 145 of the magnetic moments of the magnets 140 are disposed substantially perpendicularly to one another, i.e., rotated about 90° about the longitudinal axis of the miniature device 100. This may be useful, e.g., to assist in steering the miniature device 100 using an externally applied magnetic field. According to other embodiments (not illustrated), the vectors 145 of the magnetic moments of the magnets 140 may be parallel or antiparallel to one another.

According to some embodiments, for example as illustrated in FIG. 16 the steering portion 101 may be shaped similarly to that described above with reference to and as illustrated in FIGS. 15A and 15B, but made entirely of a magnetic material. The magnetic material may be a permanent magnet or an electromagnet, for example as described above. According to some embodiments, the steering portion 101 comprises particles, for example nanoparticles and/or microparticles, of magnetic material dispersed in a polymer matrix. In some embodiments, the matrix of polymer is an elastomer.

It will be appreciated that, for example as described above with reference to and as illustrated in any one or more of FIGS. 15A through 16, the steering portion 101 is characterized by a streamlined shape, for example facilitating motion through tortuous passageways and/or circumventing obstacles, e.g., strands of arachnoid material encountered along the spinal cord.

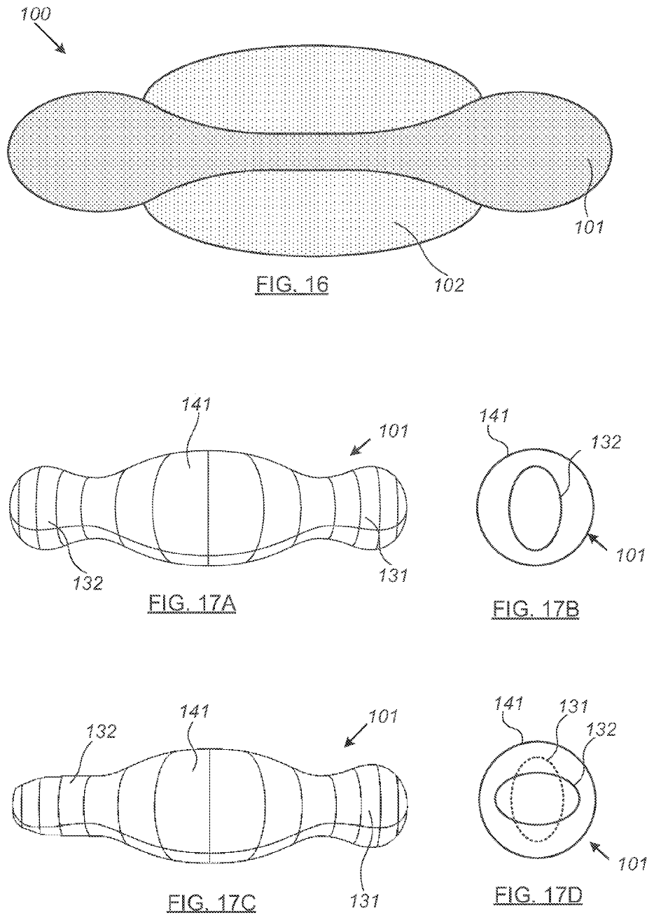

According to some embodiments, for example as illustrated in FIGS. 17A through 17D, the steering portion 101 may comprise front and rear ends 131, 132 being formed as flat shapes, for example comprising an ellipsoid-like, e.g., an oblate spheroid, shape, connected by a bridging member 141. The bridging member may be formed with a bulge, e.g., comprising an ellipsoid-like, e.g., a prolate spheroid, shape. At least the shorter dimension (seen in FIGS. 17B and 17D) of the front and rear ends 131, 132 has a smaller profile than the bridging portion 141.

According to some examples, for example as illustrated in FIGS. 17A and 17B, both of the front and rear ends 131, 132 are oriented such that their respective shorter dimensions are parallel to one another. According to other examples, for example as illustrated in FIGS. 17C and 17D, the front and rear ends 131, 132 are oriented such that their respective shorter dimensions are perpendicular to one another (the outline of the front end 131, hidden by the bridging member 141, is shown in broken lines). According to other examples (not illustrated), the front and rear ends 131, 132 are oriented such that their respective shorter dimensions are disposed at any other suitable angle with respect to one another. It will be appreciated that these examples may facilitate steering of the miniature device 100 and/or the steering portion 101 through the body.

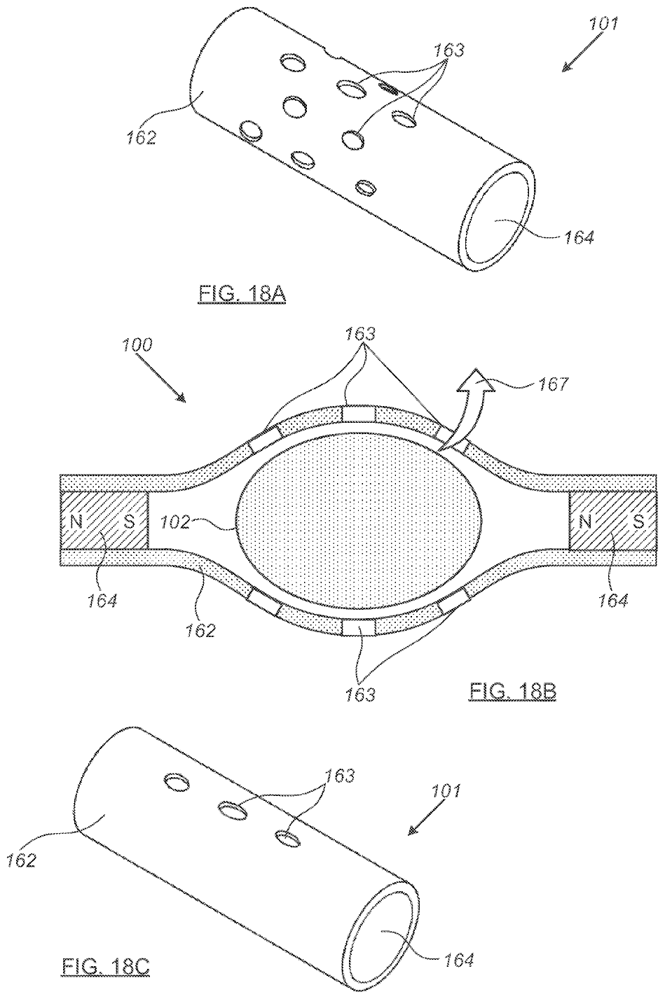

According to some embodiments, for example as illustrated in FIG. 18A and 18B, the steering portion 101 comprises a tube 162 made of elastomeric material and formed with one or more through-going aperture 163. The steering portion 101 further comprises a cap 164 disposed at each end of the tube 162, thereby closing it. One or both of the caps 164 may be or comprise a magnet, for example being a permanent magnet and/or an electromagnet as described above. The carrier portion 102 (seen in FIG. 18B) has a diameter which is larger than the tube 162, and is disposed therewithin. When the carrier portion 102 is so received within the steering portion 101, the tube 162 stretches to accommodate it and the miniature device 100 assumes a bulging state (shown in FIG. 18B).

In use, the miniature device 100 is positioned at the treatment site in a liquid environment. When the carrier portion 102 begins to dissipate, for example as described above, the therapeutic component mixes with the liquid, and the carrier portion shrinks in size, thereby releasing potential energy stored in the stretched material of the tube 162. Accordingly, the tube 162 exerts an inwardly-directed force, propelling the therapeutic component outwardly through the apertures 163, for example as illustrated by arrow 167, and returning the tube to its constricted state (as shown in FIG. 18A).

In some embodiments, the carrier portion 102 may comprise citric acid and sodium bicarbonate, resulting in an effervescent reaction.

According to some examples, the tube 162 may be formed with through-going apertures 163 around its entire circumference. According to other embodiments, for example as illustrated in FIG. 18C, the tube 162 comprises through-going apertures 163 only partially around its circumference, for example facing a single direction. This may facilitate, e.g., directing the therapeutic component to be propelled in a predetermined direction. According to other embodiments, the caps 164 may be oriented such that the vectors of their magnetic moments are aligned and parallel to one another (as illustrated), aligned and antiparallel, perpendicular, etc.

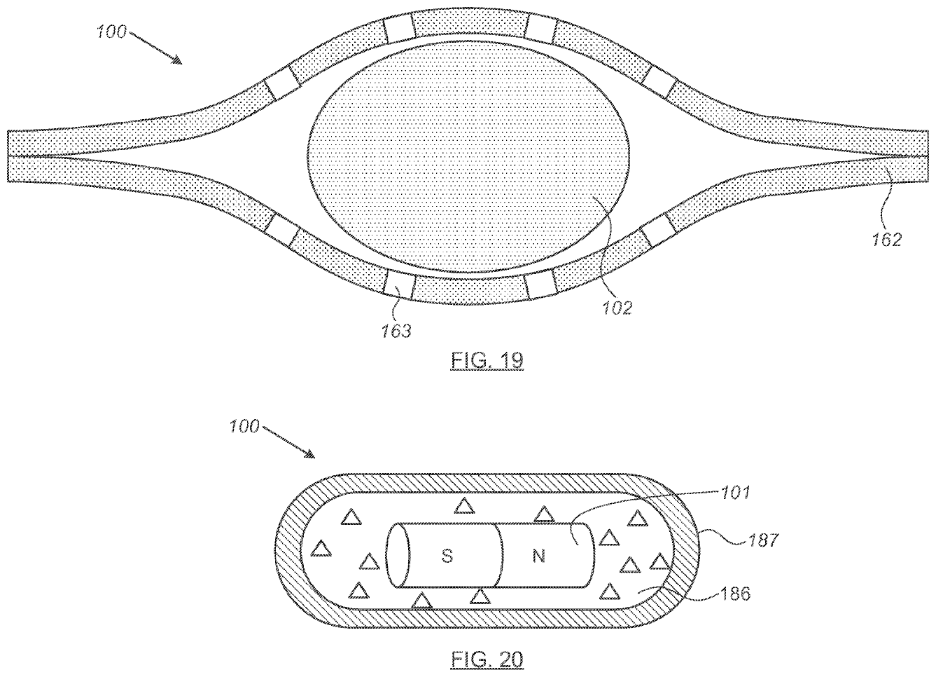

According to some embodiments, for example as illustrated in FIG. 19, the steering portion 101 may comprise a tube 162, for example as described above with reference to and as illustrated in any one or more of FIGS. 18A through 18C, with the modification that the tube is magnetic, for example being impregnated with magnetic particles, for example microparticles and/or nanoparticles.

According to some embodiments, for example as illustrated in FIG. 20, the carrier portion 102 comprises a liquid 186 disposed within a casing 187. The therapeutic component may be mixed with the liquid 186, the casing 187, or both. According to some examples, the liquid 186 may be configured to react with the material of the casing 187, thereby hastening dissolving of the casing. According to some examples, the liquid 186 is acidic, for example being bupivacaine. The miniature device 100 may be configured such that agitation thereof results in impacts of the steering portion 101 on the inner side of the casing 187, which may facilitate rupture thereof and release of the liquid 186 therefrom.

While certain features of the presently disclosed subject matter have been illustrated and described herein, many modifications, substitutions, changes, and equivalents will now occur to those of ordinary skill in the art. It is, therefore, to be understood that the appended claims are intended to cover all such modifications and changes as fall within the true spirit of the presently disclosed subject matter.

1. A miniature device for use in a system configured to deliver a therapeutic component to a treatment site in a patient, the miniature device comprising:
at least one steering portion comprising a magnetic material; and
at least one carrier portion affixed to the steering portion and comprising the therapeutic component, the carrier portion being configured to at least partially dissipate under one or more predetermined conditions at the treatment site, thereby releasing the therapeutic component.

2. The miniature device according to claim 1, wherein the carrier portion further comprises a binder material mixed with the therapeutic component and being configured to undergo the dissipation.

3. The miniature device according to claim 2, wherein the binder material comprises a biodegradable and/or a bioerodible polymer.

4. The miniature device according to claim 2, wherein the binder material comprises one or more selected from the group including polylactic acid, agar, poly(lactic-co-glycolic acid), chitosan, hyaluronic acid, a hyaluronic acid salt, gelatin, glucose, and carboxymethyl cellulose.

5. The miniature device according to any one of the preceding claims, further comprising an auxiliary carrier portion configured to at least partially dissipate under one or more predetermined conditions at the treatment site.

6. The miniature device according to claim 5, wherein the auxiliary carrier portion completely surrounds the carrier portion.

7. The miniature device according to any one of claims 5 and 6, wherein the auxiliary carrier portion comprises a therapeutic component which differs from that of the carrier portion.

8. The miniature device according to any one of claims 5 and 6, wherein the auxiliary carrier portion comprises the same therapeutic component as does the carrier portion at a different concentration.

9. The miniature device according to any one of claims 5 and 6, wherein the auxiliary carrier portion is free of a therapeutic component.

10. The miniature device according to any one of the preceding claims, wherein the carrier portion is formed with one or more channels open at an outer surface thereof and extending therewithin.

11. The miniature device according to any one of the preceding claims, wherein the carrier portion is formed with one or more chambers therewith.

12. The miniature device according to claim 11, wherein at least one of the chambers is evacuated.

13. The miniature device according to any one of claims 11 and 12, wherein at least one of the chambers comprises therewithin one or more gases selected from the group including air, hydrogen, oxygen, nitrogen, and carbon dioxide.

14. The miniature device according to any one of the preceding claims, wherein the carrier portion is affixed to the steering portion by an adhesive material.

15. The miniature device according to claim 15, wherein the adhesive material is configured to be disrupted under a predetermined condition, thereby separating the carrier portion from the steering portion.

16. The miniature device according to claim 16, wherein the predetermined condition under which the adhesive material is configured to be disrupted is one or more selected from the group including melting, dissolving in a solvent, chemically induced matrix rupture, exposure to radio and/or ultrasound waves, exposure to near infrared frequency.

17. The miniature device according to any one of claims 14 through 16, wherein the adhesive material is insulated from the environment by a bioerodible material configured to delay the disruption of the adhesive material.

18. The miniature device according to any one of the preceding claims, wherein the carrier portion surrounds the steering portion.

19. The miniature device according to any one of the preceding claims, wherein the steering portion comprises a non-magnetic shell at least partially surrounding the magnetic material, the carrier portion being at least partially affixed thereto.

20. The miniature device according to any one of the preceding claims, wherein the steering portion comprises two magnets constituting the magnetic material and being spaced along a longitudinal axis of the miniature device, the steering portion further comprising a non-magnetic bridging member spanning therebetween.

21. The miniature device according to claim 20, wherein the carrier portion is disposed surrounding the bridging member.

22. The miniature device according to any one of claims 20 and 21, wherein the vectors of the magnetic moments of the magnets are parallel to each other.

23. The miniature device according to any one of claims 20 and 21, wherein the vectors of the magnetic moments of the magnets are antiparallel to each other.

24. The miniature device according to any one of claims 20 and 21, wherein the vectors of the magnetic moments of the magnets are perpendicular to each other.

25. The miniature device according to any one of claims 20 through 24, wherein the magnets are oriented such that the vectors of their magnetic moments are perpendicular to the longitudinal axis of the miniature device.

26. The miniature device according to any one of claims 20 through 23, wherein the magnets are oriented such that the vectors of their magnetic moments are parallel to the longitudinal axis of the miniature device.

27. The miniature device according to any one of the preceding claims, wherein the miniature device is substantially shaped as a prolate spheroid.

28. The miniature device according to claim 27, being formed with an indentation at a rear end thereof, the indentation being configured to accommodate a front end of another similarly formed miniature device.

29. The miniature device according to any one of claims 1 through 18, wherein the steering portion comprises a tube made of an elastomeric materiel and being formed with one or more through-going apertures, the carrier portion being disposed within the tube and having a larger diameter than the tube.

30. The miniature device according to claim 29, wherein the steering portion further comprises a magnet closing each end of the tube.

31. The miniature device according to claim 29, wherein the tube is magnetic.

32. The miniature device according to any one of the preceding claims, wherein the carrier portion comprises a liquid and a rigid casing therearound, the rigid casing being configured to undergo the dissipation.

33. The miniature device according to claim 32, wherein the steering portion is disposed within the liquid.

34. The miniature device according to any one of the preceding claims, wherein the carrier portion comprises one or more materials configured to effervesce during the dissipation.

35. A system configured to deliver a therapeutic component to a treatment site in a patient, the system comprising at least one miniature device according to any one of the preceding claims, the system further comprising a magnetic inducing apparatus configured to be operated to generate a varying magnetic field, thereby remotely controlling motion of the miniature device.
